(19) United States
(12) Patent Application Publication
(10) Pub. No.: US 2014/0031633 A1
(43) Pub. Date: Jan. 30, 2014

(54) FOUR-VALVE VAGINAL SPECULUM

(71) Applicant: Jonathan Cohen, Paris (FR)

(72) Inventor: Jonathan Cohen, Paris (FR)

(73) Assignee: ASSISTANCE PUBLIQUE - HOPITAUX DE PARIS, Paris (FR)

(21) Appl. No.: 13/985,429

(22) PCT Filed: Feb. 3, 2012
(86) PCT No.: PCT/EP2012/051879

§ 371 Date: Oct. 10, 2013

(30) Foreign Application Priority Data
Feb. 14, 2011 (FR) 1151188

(51) Int. Cl. A61B 1/303; A61B 1/32

(52) U.S. Cl. CPC A61B 1/303; A61B 1/32
USPC 600/231

(57) ABSTRACT

A vaginal speculum includes a first rod supporting a first arm that is slidable along the first rod, a first valve being attached to the first arm, and a second arm, a second valve being attached to the second arm. The vaginal speculum further includes a second rod supporting a third arm that is slidable along the second rod, a third valve being attached to the third arm, and a fourth arm, a fourth valve being attached to the fourth arm. The first rod and the second rod are rigidly connected to one another.

### TECHNICAL FIELD OF THE INVENTION

The present invention relates to a four-valve vaginal speculum.

The technical field is generally that of vaginal valve devices intended to move the vagina walls apart. It is particularly suitable for an immediate post-partum vaginal exploration or vaginal surgery, that is within twenty four hours of a delivery. However, it can also be used besides post-partum in any kind of vaginal surgery, for example a hysterectomy through the vaginal route, cerclage, conization, laser vaporisation of the cervix, prolapse surgery or even suturing of vaginal wounds.

The speculum according to the invention is used in particular, but not exclusively, in case of vaginal haemorrhage to search for a cervical or vaginal injury responsible for bleeding. It is an essential examination which should not be disregarded, in order not to unnecessarily or inefficiently treat an uterine cause of the haemorrhage.

### STATE OF PRIOR ART

Currently, an obstetrician performing an immediate post-partum vaginal exploration or vaginal surgery is helped by an assistant who keeps two vaginal valves on either side of the vagina walls. But, in case of emergency, an assistant is not always available.

On the other hand, Cusco™ or Collin™ type vaginal specula are not suitable for immediate post-partum. These specula include two arms joined together by an end, each arm being rectilinear and having an arcuate cross-section so as to have a convex outer surface and a concave inner surface, means being provided to keep an angular deviation between both arms. These arms are generally called valves. The obstetrician inserts them into the vagina during an examination before increasing their angular deviation. But in immediate post-partum, the vagina has undergone a high distension. The dimensions of these specula are then no longer suitable for keeping the walls of the distended vagina sufficiently apart, nor to be kept alone in position in the vagina.

### GENERAL DESCRIPTION OF THE INVENTION

The object of the invention provides a vaginal valve instrument able to move vaginal walls apart, in particular in immediate post-partum, and to keep them in position without external intervention.

The invention thus substantially relates to a vaginal speculum including:
a first rod supporting:
   a first arm slidable along the first rod, a first valve being attached to the first arm;
   a second arm, a second valve being attached to the second arm;
a second rod supporting:
   a third arm slidable along the second rod, a third valve being attached to the third arm;
   a fourth arm, a fourth valve being attached to the fourth arm;

the first rod and the second rod being integral with each other.

An obstetrician using the vaginal speculum according to the invention to perform a vaginal exploration or a vaginal surgery, places the four valves inside the vagina, and moves them apart by virtue of the ability of the first arm and the third arm to slide along the first rod and the second rod, respectively, thus moving the vagina walls apart.

By virtue of the invention, the vaginal walls are kept apart, and the speculum is kept alone in position in the vagina. An assistant is therefore not necessary, and the instrument is usable in immediate post-partum.

Besides the characteristics just discussed in the preceding paragraph, the vaginal speculum according to the invention can have one or more further characteristics from the following ones, considered singly or in accordance with any technically possible combinations:

the first valve and the second valve are planar and extend along planes tilted each other at an angle between 0 and 30 degrees, and preferably between 5 and 20 degrees. Such an arrangement improves keeping the speculum according to the invention on the vagina.

The third valve and the fourth valve extend along planes tilted each other at an angle between 0 and 30 degrees, and preferably between 5 and 20 degrees. Such an arrangement improves keeping the speculum according to the invention on the vagina.

The first rod is provided with a first rack system able to slide the first arm along the first rod. Such an arrangement is particularly convenient for an obstetrician using the speculum according to the invention. Indeed, the first arm is freely slidable along the first rod in a direction wherein the first valve and the second valve move apart from each other, whereas a sliding motion in the reverse direction is blocked, advantageously by a check valve.

The second rod is provided with a second rack system able to slide the third arm along the second rod. Such an arrangement is particularly convenient for an obstetrician using the speculum according to the invention. Indeed, the third arm is freely slidable along the second rod in a direction wherein the third valve and the fourth valve move apart from each other, whereas a sliding motion in the reverse direction is blocked, advantageously by a check valve.

The vaginal speculum includes a first means for setting the position of the first arm to the first rod. Thus, an obstetrician using the speculum according to the invention to keep the vagina walls apart, is able, after inserted the four valves into the vagina, and determining an optimum deviation between the first valve and the second valve, to maintain this deviation by setting the position of the first arm to the first rod.

The vaginal speculum includes a second means for setting the position of the third arm on the second rod. Thus, an obstetrician using the speculum according to the invention to keep the vagina walls apart, is able, after inserted the four valves into the vagina, and determining an optimum deviation between the third valve and the fourth valve, to maintain this deviation by setting the position of the first arm to the first rod.

The first rod and the second rod extend along intersected, and preferably substantially orthogonal, directions. Such an arrangement is particularly advantageous to improve keeping the speculum according to the invention on the vagina.

The first arm and the first rod extend along substantially orthogonal directions. Such an arrangement is particularly advantageous to improve keeping the speculum according to the invention on the vagina.

The second arm and the first rod extend along substantially orthogonal directions. Such an arrangement is particularly advantageous to improve keeping the speculum according to the invention on the vagina.

The third arm and the second rod extend along substantially orthogonal directions. Such an arrangement is particularly advantageous to improve keeping the speculum according to the invention on the vagina.

The fourth arm and the second rod extend along substantially orthogonal directions. Such an arrangement is particularly advantageous to improve keeping the speculum according to the invention on the vagina.

According to one embodiment, the vaginal speculum according to the invention is of metal. In this embodiment, the speculum is robust and reusable.

According to another embodiment, the vaginal speculum according to the invention is of plastics. In this embodiment, the speculum is for single use and advantageously presterilized. It does not need to be sterilized before each examination.

According to one embodiment, the valves are solid. Solid valves enable the vaginal walls to be compressed in order to perform a possible mechanical haemostasis of a vaginal bleeding caused by a spontaneous tear or by an episiotomy.

According to another embodiment, the valves are hollow. Hollow valves provide for a greater visibility on the inner walls of the vagina. They are more suitable for performing suturing of the vaginal mucosa if need be.

The invention and the different applications thereof will be better understood upon reading the following description and examining the accompanying figures.

### BRIEF DESCRIPTION OF THE FIGURES

The figures are only set forth by way of illustrating and in no way limiting purpose of the invention. The figures show:

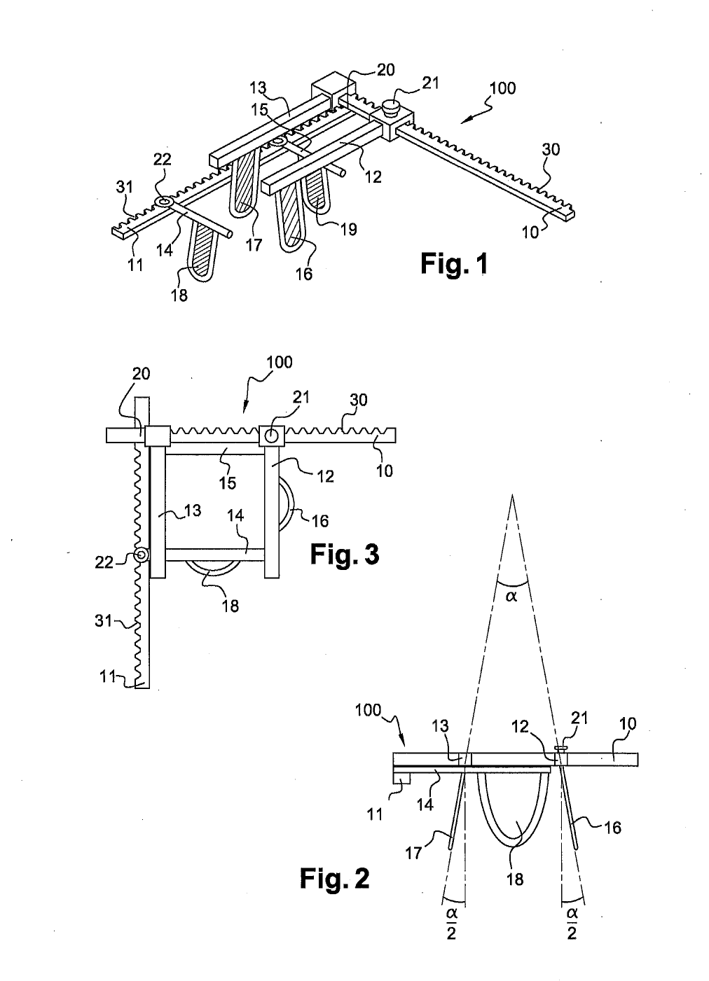

in FIG. 1, a perspective view of a vaginal speculum according to one embodiment of the invention;

in FIG. 2, a front view of said speculum;

in FIG. 3, a top view of said speculum.

### DETAILED DESCRIPTION OF AT LEAST ONE EMBODIMENT OF THE INVENTION

Unless otherwise mentioned, a same element appearing in different figures has a single reference.

FIG. 1 is a perspective view of a vaginal speculum 100 having four valves according to a non-limiting embodiment of the invention.

The vaginal speculum 100 includes:
a first rod 10
a second rod 11
a means 20 for securing the first rod 10 and the second rod 11
a first arm 12
a second arm 13
a third arm 14
a fourth arm 15
a first valve 16
a second valve 17
a third valve 18
a fourth valve 19
a first means 21 for setting the position of the first arm 12 to the first rod 10
a second means 22 for setting the position of the third arm 14 to the second rod 11
a first rack system 30
a second rack system 31.

The first rod 10 and the second rod 11 are integral with each other by virtue of the securing means 20. The securing means 20 is for example a weld if the vaginal speculum 100 is made of metal. The securing can also be achieved by screwing, or even bonding, in particular if the vaginal speculum 100 is of plastics. In another embodiment of the invention, the first rod 10 and the second rod 11 are made as one piece.

The first arm 12 and the second arm 13 are supported by the first rod 10. In the non-limiting embodiment described, the first arm 12 and the second arm 13 extend along parallel directions, and said direction is orthogonal to the direction along which the first rod 10 extends. The third arm 14 and the fourth arm 15 are supported by the second rod 11. In the non-limiting embodiment described, the third arm 14 and the fourth arm 15 extend along parallel directions, and said direction is orthogonal to the direction along which the second rod 11 extends.

The first valve 16, the second valve 17, the third valve 18 and the fourth valve 19 are respectively attached to the first arm 12, the second arm 13, the third arm 14 and the fourth arm 15. Two types of valves are defined: solid valves and hollow valves. A valve consists of a closed perimeter and an inner space, the inner space being bounded by the perimeter. When the inner space is empty, this is called an empty valve, and when the inner space is integrally made of material, this is called a solid valve. In the non-limiting embodiment described, the four valves are solid, but according to other embodiments, at least one valve is hollow.

The first arm 12 is slidable along the first rod 10. The first means 21 for setting the position of the first arm 12 to the first rod 10 is able to block the sliding motion of the first arm 12 along the first rod 10. Furthermore, the third arm 14 is slidable along the second rod 11. The second means 22 for setting the position of the third arm 14 to the second rod 11 is able to block the sliding motion of the third arm 14 along the second rod 11.

In another embodiment of the invention, the second arm 13 is also slidable along the first rod 10, and the fourth arm 15 is also slidable along the second rod 11.

FIG. 2 is a top view of a vaginal speculum 100. In the embodiment described, the valves are planar and extend in planes tilted with respect to each other at an angle α. According to a non-limiting embodiment of the invention, the angular deviation between the plane along which the first valve 16 extends, and a plane orthogonal to the direction along which the first rod 10 extends, is α/2. The angular deviation between the plane along which the second valve 17 extends, and a plane orthogonal to the direction along which the first rod 10 extends, then is also α/2. α is advantageously between 0 and 30 degrees, and preferably between 5 and 20 degrees. In the non-limiting embodiment represented in FIG. 2, α is 20°.

FIG. 3 is a top view of a vaginal speculum 100. The first rod 10 is provided with a first rack system 30, and the second rod 11 is provided with a second rack system 31. Such a system is particularly convenient for an obstetrician using the vaginal speculum 100 according to the invention. Indeed, by virtue of this system, the first arm 21 is freely slidable along the first rod 10 in a direction wherein the first valve 16 and the second valve 17 move apart from each other, and the third arm 14 is freely slidable along the second rod 11 in a direction wherein the third valve 18 and the fourth valve 19 move apart from each other, whereas the sliding motions in the reverse directions are blocked. These sliding motions in reverses directions are blocked for example by two check valves, and unblocked when the obstetrician actuates the check valves.

In such an embodiment, both check valves make up the first means 21 for setting the position of the first arm 12 to the first rod 10 and the second means 22 for setting the position of the third arm 14 to the second rod 11.

An obstetrician using the vaginal speculum 100 for performing an immediate post-partum vaginal exploration or vaginal surgery, places the four valves inside the vagina, and moves them apart to each other by virtue of the ability of the first arm and the third arm to slide along the first rod and the second rod, respectively, thus moving apart the vagina walls. When the obstetrician has determined a deviation of the valves that suits him (her), he/she sets the position of the valves by the first means 21 for setting the position of the first arm 12 to the first rod 10 and the second means 22 for setting the position of the third arm 14 to the second rod 11. The vaginal speculum 100 is then kept in position in the vagina without external intervention, and therefore the obstetrician has available both his/her hands to perform a vaginal exploration or a vaginal surgery.

1. A vaginal speculum comprising:
a first rod supporting:
a first arm slidable along the first rod, a first valve being attached to the first arm;
a second arm, a second valve being attached to the second arm;
a second rod supporting:
a third arm slidable along the second rod, a third valve being attached to the third arm;
a fourth arm, a fourth valve being attached to the fourth arm;
the first rod and the second rod being integral with each other.

2. The vaginal speculum according to claim 1, wherein the first valve and the second valve are planar and extend along planes tilted to each other at an angle a between 0 and 30 degrees.

3. The vaginal speculum according to claim 1, wherein the third valve and the fourth valve extend along planes tilted to each other at an angle α between 0 and 30 degrees.

4. The vaginal speculum according to claim 1, wherein:
the first rod is provided with a rack system able to slide the first arm along the first rod;
the second rod is provided with a second rack system able to slide the third rod along the second rod.

5. The vaginal speculum according to claim 1, comprising:
a first means for setting the position of the first arm to the first rod;
a second means for setting the position of the third arm to the second rod.

6. The vaginal speculum according to claim 1, wherein the first rod and the second rod extend along intersecting, directions.

7. The vaginal speculum according to claim 1, wherein:
the first arm and the first rod extend along substantially orthogonal directions;
the second arm and the first rod extend along substantially orthogonal directions;
the third arm and the second rod extend along substantially orthogonal directions;
the fourth arm and the second rod extend along substantially orthogonal directions.

8. The vaginal speculum according to claim 1, it wherein the speculum is made of metal.

9. The vaginal speculum according to claim 1, it wherein the speculum is made of plastics.

10. The vaginal speculum according to claim 1, wherein the valves are solid.

11. The vaginal speculum according to claim 1, wherein the valves are hollow.

12. The vaginal speculum according to claim 2, wherein the angle α is between 5 and 20 degrees.

13. The vaginal speculum according to claim 3, wherein the angle α is between 5 and 20 degrees.

14. The vaginal speculum according to claim 5, wherein the first and second means include a check valve.

15. The vaginal speculum according to claim 6, wherein the first rod and the second rod extend along substantially orthogonal directions.
